(19) United States
(12) Patent Application Publication
(10) Pub. No.: US 2024/0197556 A1
(43) Pub. Date: Jun. 20, 2024

(54) SYSTEMS, DEVICES AND METHODS FOR ENACTING CYCLICAL POSITIVE AND NEGATIVE PRESSURE

(71) Applicant: Vikas Khurana, South Abington Township, PA (US)

(72) Inventor: Vikas Khurana, South Abington Township, PA (US)

(21) Appl. No.: 18/286,367

(22) PCT Filed: Apr. 11, 2022
(86) PCT No.: PCT/US2022/024216

§ 371 Date: Oct. 11, 2023

Related U.S. Application Data
(60) Provisional application No. 63/201,076, filed on Apr. 11, 2021.

(51) Int. Cl. A61H 9/00

(52) U.S. Cl. CPC A61H 9/0078; A61H 2201/1246; A61H 2201/1409; A61H 2201/164; A61H 2201/165; A61H 2201/5007; A61H 2201/5053; A61H 2201/5056; A61H 2201/5074; A61H 2203/0468; A61H 2205/106; A61H 2209/00; A61H 2230/045; A61H 2230/255

(57) ABSTRACT

Disclosed a system (100) for enacting a positive and a negative pressure on a limb. The system (100) includes an air compressor (102) coupled to a reservoir (104), a piston assembly (106) configured to the reservoir (104), wherein the piston assembly (106) further comprises a piston (108) circumfused inside a tube (107), a pneumatic ring (114) coupled to the system (100) via the piston assembly (106) maintains intermittent pressure and prevents formation of a venous blood clot ischemic injury to a tissue distal to the pneumatic ring (114) compression due to lack of blood flow.

### TECHNICAL FIELD

The present disclosure relates to systems, devices, and methods for creating cyclical positive and negative pressure on and/or inside biological or non-biological systems.

### BACKGROUND

It is known that as of now, there are several methodologies to enact on a positive or a negative pressure however, the same are not available in a cyclical fashion.

The cyclical generation of positive and negative pressure is required at numerous sites and conditions e.g., in biological systems, more particularly in humans. For example, there are times when a cycling negative and positive pressure is needed to execute an outcome for example, once a blood vessel is blocked and the blood flow to the distal area is compromised, the tissue injury sets in causing edema. The edema does not allow adequate ultra-filtration and microcirculation as additional forces required to overcome the edema are being forced upon the tissue. This leads to a complicated environment where if only a negative force is applied the edema further worsens. A positive force will drain the edema but will not increase the blood flow to the area leading to a short-term relief of edema but no healing. Thus, the positive and negative cycling of pressures are beneficial as the positive pressure would remove the edema and the negative pressure brings new blood to the area.

Further, it is known that Galvanic current of an inflamed area is different than a healthy area. In patients with hypoxic injury due to ischemia secondary to microvascular disease, when once corrected, there is a difference in the galvanic current. The difference is thus used to demonstrate clinical improvement in the patient.

Therefore, a need for devices, systems, and methods for generating cyclical positive and negative pressure persists, so that the same may be applied at a desired site of any subject.

### SUMMARY OF THE INVENTION

One aspect of the present disclosure provides a system for enacting a positive and a negative pressure on a limb. The system includes a reservoir to entrap air. The system further includes an air compressor coupled to a reservoir to entrain atmospheric air. The system further includes a piston assembly configured to the reservoir wherein a forward movement of the piston enacts positive pressure, and a backward movement of the piston enacts negative pressure. Further, a pneumatic ring is coupled to the system via the piston assembly to maintain intermittent pressure which prevents the formation of a venous blood clot and ischemic injury to a tissue distal to the pneumatic ring compression due to lack of blood flow.

In some aspect of the present disclosure, the system further includes a pressure measuring mechanism selected from a group, but not limited to, a pressure gauge, barometer, piezometer, pressure tube, manometer, bourdon gauge, diaphragm pressure gauge, micro manometer.

In some aspect of the present disclosure, the pressure enacted by the system is sensed by the pneumatic ring placed over a portion of the limb.

In some aspect of the present disclosure, the system further includes a dual piston assembly to enact positive and negative pressure on the limb with one or both of a first piston assembly and a second piston assembly. The system includes the air compressor coupled to the reservoir. Further, a pneumatic chamber assembly is coupled to the system to maintain intermittent pressure to prevent formation of a blood clot. The first piston assembly further includes a first piston, and the second piston assembly includes a second piston. The forward movement of the first piston, towards the limb, enacts positive pressure and the backward movement of the second piston, away from the limb, enacts negative pressure.

In some aspect of the present disclosure, the system further includes a plurality of reservoirs, wherein the first piston assembly is connected to a first reservoir and the second piston assembly is connected to a second reservoir.

In some aspect of the present disclosure, the first piston assembly moves in forward direction, towards the limb, to enact positive pressure whereas the second piston assembly moves in backward direction, away from the limb, to enact negative pressure.

In some aspect of the present disclosure, the positive and negative pressure enacted by the system is sensed by the pneumatic chamber assembly placed over a portion on the limb via plurality of solenoid valves.

In some aspect of the present disclosure, the pressure enacted by the system is sensed by the pneumatic chamber assembly through a first solenoid valve and the excess pressure is released into the environment through a second solenoid valve.

Another aspect of the present disclosure provides a method for treating a damaged tissue by providing positive and negative pressure. The method includes enacting positive and negative pressure by way of forward and backward motion of the piston, where the forward movement of the piston creates positive pressure, and the backward movement of the piston creates negative pressure. The method further includes altering a proximal artery compression to increase intra-arterial pressure. The method further includes modulating pressure in a pneumatic chamber assembly and increasing shear forces on an endothelial cell to remodel the endothelium and vasculature.

In some aspect of the present disclosure, the method further includes a step for relieving arterial stiffness by increasing dilation in the artery.

In some aspect of the present disclosure, the method further increases the oxygen transfer rate in an arterial system.

Another aspect of the present disclosure provides a system for enacting positive and negative pressure on a limb. The system includes a bellow coupled to a solenoid actuator. The system further includes a pneumatic chamber assembly coupled to the system to maintain intermittent pressure to prevent formation of a venous blood clot. The system enacts positive and negative pressure by the action of the solenoid actuator on bellow. The solenoid actuator compresses the bellow to enact positive pressure and decompresses the bellow to enact negative pressure.

In some aspect of the present disclosure, the pressure enacted by the system is sensed by the pneumatic chamber assembly via a first solenoid valve and the excess pressure is released into the environment via a second solenoid valve.

In some aspect of the present disclosure, the system further includes an assembly of bellows to enact positive and negative pressure on the limb, with one of a first bellow and a second bellow. The first bellow is connected to a first solenoid actuator and the second bellow is connected to a second solenoid actuator. The pneumatic chamber assembly is further coupled to the system to maintain intermittent pressure to prevent venous blood clot formation. The system enacts positive and negative pressure by the action of the solenoid actuator on bellow. The first solenoid actuator compresses the first bellow to enact positive pressure and the second solenoid actuator decompresses the second bellow to enact negative pressure.

Another aspect of the present disclosure provides a method for treating a damaged tissue by providing positive and negative pressure. The method includes enacting positive and negative pressure by action of a bellow. A solenoid actuator compresses the bellow to enact positive pressure and the solenoid actuator decompresses pressure on the bellow to enact negative pressure. The method further includes altering a proximal artery compression, modulating pressure in a pneumatic chamber assembly to increase intra-arterial pressure, and increasing shear forces on an endothelial cell to remodel the endothelium and vasculature.

Another aspect of the present disclosure provides a system for enacting positive and negative pressure on a limb with one or both of a first piston assembly and a second piston assembly. The system includes an air compressor coupled to a reservoir. The first piston assembly further includes a first piston, and the second piston assembly includes a second piston. A pneumatic chamber assembly is coupled to the system to maintain intermittent pressure and prevent formation of a venous blood clot. The forward movement of the first piston, towards the limb, enacts positive pressure and the backward movement of the second piston, away from the limb, enacts negative pressure.

Another aspect of the present disclosure provides a system for enacting positive and negative pressure on a limb with one or both of a first bellow and a second bellow. The system includes a first bellow that is connected to a first solenoid actuator and the second bellow that is connected to a second solenoid actuator. A pneumatic chamber assembly is further coupled to the system to maintain intermittent pressure and prevent venous blood clot formation. The system enacts positive and negative pressure by the action of the solenoid actuator on bellow. The first solenoid actuator compresses the first bellow to enact positive pressure and the second solenoid actuator decompresses the second bellow to enact negative pressure.

Another aspect of the present disclosure provides a system for enacting positive and negative pressure on a skin with one or both of a first piston assembly and a second piston assembly. The system includes an air compressor coupled to a reservoir. The first piston assembly further includes a first piston, and the second piston assembly includes a second piston. A pneumatic chamber assembly is coupled to the system to maintain intermittent pressure and prevent formation of a venous blood clot. The forward movement of the first piston, towards the limb, enacts positive pressure and the backward movement of the second piston, away from the limb, enacts negative pressure.

In some aspect of the present disclosure, the system further includes a plurality of reservoirs, wherein the first piston assembly is connected to a first reservoir and the second piston assembly is connected to a second reservoir.

In some aspect of the present disclosure, the first piston assembly moves in forward direction, towards the limb, to enact positive pressure whereas the second piston assembly moves in backward direction, away from the limb, to enact negative pressure.

In some aspect of the present disclosure, the positive and negative pressure enacted by the system is sensed by the pneumatic chamber assembly placed over a portion on the limb via plurality of solenoid valves.

In some aspect of the present disclosure, the pressure enacted by the system is sensed by the pneumatic chamber assembly through a first solenoid valve and the excess pressure is released into the environment through a second solenoid valve.

Another aspect of the present disclosure provides a method for treating edema. The method includes placing a pneumatic chamber assembly on the edema. Enacting a positive and a negative pressure by way of forward and backward motion of a first piston and a second piston, wherein the forward movement of the first piston enacts a positive pressure, and the backward movement of the second piston enacts a negative pressure in a pneumatic chamber assembly. The method further includes modulating pressure in pneumatic chamber assembly based on pulse cycle of a subject and increasing shear forces on an endothelial cell to remodel the endothelium and vasculature to treat the damaged tissue.

Another aspect of the present disclosure provides a method for treating a bed sore. The method includes placing a pneumatic chamber assembly on the edema. Enacting a positive and a negative pressure by way of forward and backward motion of a first piston and a second piston, wherein the forward movement of the first piston enacts a positive pressure, and the backward movement of the second piston enacts a negative pressure in a pneumatic chamber assembly. The method further includes modulating pressure in pneumatic chamber assembly based on pulse cycle of a subject and increasing shear forces on an endothelial cell to remodel the endothelium and vasculature to treat the damaged tissue.

Another aspect of the present disclosure provides a system for enacting positive and negative pressure on skin with one or both of a first bellow and a second bellow. The system includes a first bellow that is connected to a first solenoid actuator and the second bellow that is connected to a second solenoid actuator. A pneumatic chamber assembly is further coupled to the system to maintain intermittent pressure and prevent venous blood clot formation. The system enacts positive and negative pressure by the action of the solenoid actuator on bellow. The first solenoid actuator compresses the first bellow to enact positive pressure and the second solenoid actuator decompresses the second bellow to enact negative pressure.

In some aspect of the present disclosure, wherein the pressure enacted by the system is sensed by the pneumatic chamber assembly via a first solenoid valve and the excess pressure is released into the environment via a second solenoid valve.

Another aspect of the present disclosure provides a method for treating edema. The method includes placing a pneumatic chamber assembly on the edema. enacting a positive and negative pressure, wherein the first solenoid actuator compresses the first bellow to enact positive pressure, and the second solenoid actuator decompresses the second bellow to enact negative pressure. The method further includes modulating pressure in pneumatic chamber assembly based on pulse cycle of a subject and increasing shear forces on an endothelial cell to remodel the endothelium and vasculature to treat the damaged tissue.

Another aspect of the present disclosure provides a method for treating a bed sore. The method includes placing a pneumatic chamber assembly on the edema. enacting a positive and negative pressure, wherein the first solenoid actuator compresses the first bellow to enact positive pressure, and the second solenoid actuator decompresses the second bellow to enact negative pressure. The method further includes modulating pressure in pneumatic chamber assembly based on pulse cycle of a subject and increasing shear forces on an endothelial cell to remodel the endothelium and vasculature to treat the damaged tissue.

### BRIEF DESCRIPTION OF DRAWINGS

The drawing/s mentioned herein disclose exemplary embodiments of the claimed invention. Other objects, features, and advantages of the present invention will be apparent from the following description when read with reference to the accompanying drawing.

FIG. 1A illustrates a system having a piston to create a cyclical positive and negative pressure, according to an embodiment herein;

FIG. 1B illustrates a pneumatic assembly, according to an embodiment herein;

FIG. 2 illustrates a system and/or device having dual piston to create a cyclical positive and negative pressure, according to an embodiment herein;

FIG. 3 illustrates a flowchart that depicts the method for treating a damaged tissue by providing positive and negative pressure using a piston assembly, according to an embodiment herein;

FIG. 4 illustrates a system and having an assembly of two bellows to create a cyclical positive and negative pressure, according to an embodiment herein;

FIG. 5 illustrates a system having a bellow to create a cyclical positive and negative pressure, according to an embodiment herein;

FIG. 6 illustrates a flowchart that depicts the method for treating a damaged tissue by providing positive and negative pressure using a bellow, according to an embodiment herein;

FIG. 7A illustrates a system and/or device having dual piston to create a cyclical positive and negative pressure, according to an embodiment herein;

FIG. 7B illustrates a dome-shaped pneumatic assembly, according to an embodiment herein;

FIG. 8 illustrates a system and/or device having an assembly of two bellows to create a cyclical positive and negative pressure, according to an embodiment herein;

FIG. 8B illustrates a dome-shaped pneumatic assembly, according to an embodiment herein;

To facilitate understanding, like reference numerals have been used, where possible to designate like elements common to the figures.

### DETAILED DESCRIPTION OF PREFERRED EMBODIMENTS

This section is intended to provide explanation and description of various possible embodiments of the present invention. The embodiments used herein, and the various features and advantageous details thereof are explained more fully with reference to non-limiting embodiments illustrated in the accompanying drawing/s and detailed in the following description. The examples used herein are intended only to facilitate understanding of ways in which the embodiments may be practiced and to enable the person skilled in the art to practice the embodiments used herein. Also, the examples/embodiments described herein should not be construed as limiting the scope of the embodiments herein.

The term “enacts”, “builds” and “creates” are interchangeably used across the context.

As used herein “pneumatic ring” is defined as a ring placed in the proximal part of the limb to prevent the blood from flowing backwards when the compression is being done or alteration in the pressure is being enacted. In addition, it may not be there constantly as the blood flow in the veins will be impacted leading to clot formation in the veins.

As used herein, a bladder is similar to a chamber or a wider pneumatic ring assembly that encircles the limb like a blood pressure recording cuff and is capable of only providing positive pressure enactment. It still requires the negative pressure generated by the piston or the bellow to rapidly deflate it to bring the pressure enacted on the limb by the bladder to near zero.

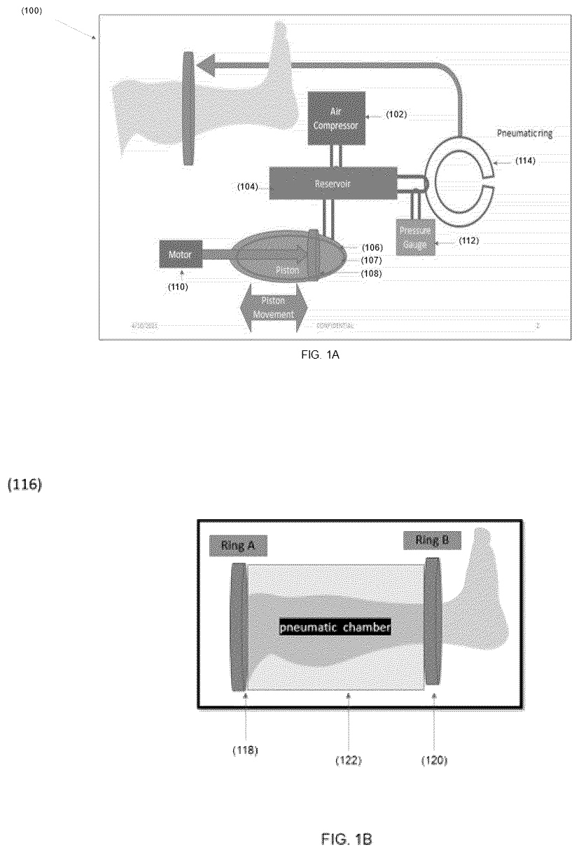

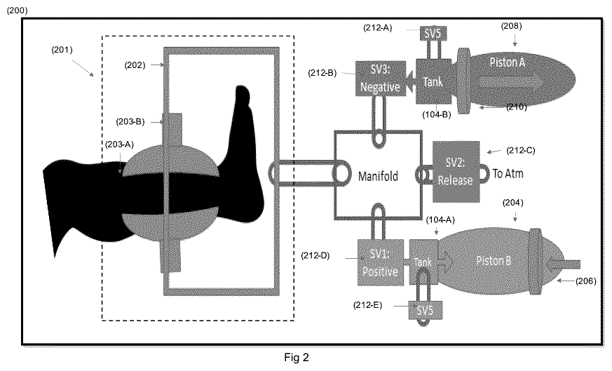

FIG. 1 illustrates a system (100) having a piston to create a cyclical positive and negative pressure, according to an embodiment herein.

The system (100) includes an air compressor (102), a reservoir (104), a piston assembly (106), a motor (110), a pressure gauge (112) and a pneumatic ring (114).

The reservoir (104) is configured to entrap air.

The air compressor (102) is coupled to the reservoir (104). The air compressor (102) entrains atmospheric air and pushes into the reservoir (104). In an embodiment, the air compressor (102) is selected from a group, but not limited to, a positive displacement air compressor, a rotatory vane compressor, a rotatory screw compressor, a reciprocating compressor, a single stage compressor, a two-stage compressor, an axial compressor, or a centrifugal compressor.

The piston assembly (106) is coupled with the reservoir (104). The piston assembly (106) includes a piston (108) circumfused inside a tube (107). The movement of the piston (108) inside the piston assembly (106) is controlled by the motor (110). The movement of the piston (108) enacts a positive and a negative pressure. When the piston (108) is positioned at the center of the tube (107), a neutral position with respect to pressure is achieved. The movement of the piston (108) in forward direction creates positive pressure, whereas the backward movement of the piston (108) creates negative pressure.

The pressure created by the piston (108) is sensed by the pneumatic ring (114), coupled to the system (100) via the piston assembly (106). The pneumatic ring (114) placed over a portion of a limb maintains intermittent pressure which prevents formation of venous blood clots and ischemic injury to the tissue distal to the pneumatic ring (114) compression due to lack of blood flow. The presence of constant pressure blocks the vein and the blood flow for too long, leading to formation of clots in the veins and further causing deep vein thrombosis. Whereas, the intermittent pressure prevents formation of venous blood clot as it enhances the flow of arterial blood to the walls of the veins thereby improving vascular health and thus decreasing the risk of formation of deep vein thrombosis.

In an embodiment, the pneumatic ring (114) is placed above knee. In another embodiment, the pneumatic ring (114) is placed below knee. In another embodiment, the pneumatic ring (114) is placed on thigh.

The pressure gauge (112) is coupled to the reservoir (104) for measuring air pressure. In an embodiment, the pressure gauge (112) is selected from a group comprising, but not limited to, a bourdon tube pressure gauge, a diaphragm pressure gauge, a capsule pressure gauge, an absolute pressure gauge, a differential pressure gauge, a piezometer pressure gauge, or a manometer pressure gauge. In another embodiment, the pressure measuring mechanism is selected from a group, but not limited to, a pressure gauge, barometer, piezometer, pressure tube, manometer, bourdon gauge, diaphragm pressure gauge, micro manometer.

In an embodiment, the pressure gauge (112) is further communicatively coupled to a processing circuitry wherein a blood flow sensor senses the blood flow and transmits to the processor. The processor further calculates the blood pressure, heart rate and displays the output on an output engine.

In an embodiment, the piston (108) is attached to the pneumatic ring (114) forming a closed-circuit loop. The action of the piston (108) inside the tube (107) builds a positive and a negative pressure. When the piston (108) is at the center of the tube (107), a neutral position is achieved in respect to pressure. The movement of the piston (108) in forward direction, towards the limb, builds positive pressure while the backward movement of the piston (108), away from the limb, builds negative pressure. The pressure built by action of the piston (108) is sensed by the pneumatic ring (114), which is placed or covering any portion of the limb.

In an embodiment, the system (100) further includes a pneumatic assembly (116), as illustrated in FIG. 1B. The pneumatic assembly (116) includes a first pneumatic ring (118), a second pneumatic ring (120) and a chamber (122). In an embodiment, the pneumatic assembly (116) is configured in such a way that it encircles the limb. During positive pressure, the pressure on the first pneumatic ring (118) is increased after the peak of the arterial pulse to further increase the flow of the blood to the feet and the chamber is pressurized to push the fluid out into the arterial system while no pressure is applied on the second pneumatic ring (120). Whereas no pressure is applied on the first and second pneumatic ring during the cycle of negative pressure and is allowed to circle only in the chamber (122) to enhance the flow. The chamber (122) completely covers the first pneumatic ring (118) and the second pneumatic ring (120), for providing positive and negative pressure to the limb. The pressure required to block the venous blood flow is significantly lower than the pressure required to block the arterial blood flow. The intermittent pressure maintained by the pneumatic ring (118) in combination with the pneumatic chamber (122) prevents formation of venous blood clot as it enhances the flow of arterial blood to the wall of veins thereby improving vascular health and thus decreasing the risk of formation of deep vein thrombosis.

In an exemplary embodiment, a dome shaped (like a bell jar) pneumatic assembly is provided for treatment of bed sores using positive and negative pressure. The pneumatic assembly is placed over an ulcer in such a way that, a holding plate pushes the skin inside the dome while the pneumatic ring holds the skin and modulates the positive and negative pressure moving under the holding plate. The pneumatic ring distributes the pressure to prevent a point area of high pressure which has a high likelihood of damaging the already compromised skin around the ulcer. The dome shaped of the assembly forms a chamber for modulating the positive and negative pressure provided to the system. The dome shaped pneumatic assembly may be further configured to a dual piston assembly or a bellow-based system to enact cyclical positive and negative pressure.

In an embodiment, the positive and negative pressure enacted by the system (100) may be sensed by a bladder and a pneumatic ring (114). In an embodiment, the system (100) further includes a bladder placed adjacent to the pneumatic ring (114). The movement of the piston (108) in forward direction, towards the limb, builds positive pressure while the backward movement of the piston (108), away from the limb, builds negative pressure, thus the positive and negative pressure enacted by the piston is sensed by the bladder and the pneumatic ring (114) respectively.

Further, in an embodiment, the cycling of positive and negative pressure may be timed with the pulse wave of the subject. There may be gaps of one or more than one heartbeat cycles to synchronize the cycling of pressures with the pulse wave. The positive pressure will decrease edema and the negative pressure will increase blood flow. The pressure modulation enacted by system (100) includes intermittent break in pressure modulation to let the tissue equilibrate and normalize. Thus, a corrective waveform is added to the underlying diseased waveform of the patient. The system (100) is used to inflate the system and as the air bleeds out of the system, the pump replaces the air. The final movement of air is controlled by the piston (108), which further controls the pressure within the bladder. In an embodiment, a variable pressure using the piston (108) in air lock may be generated.

It is further found that the pressure in the bladder or pneumatic ring (114) should be below the systolic pressure. If the pressure is higher than the systolic pressure, it induces pain. The pressure thus starts rising from each individual compression from 60% of the systolic pressure to a 100% of the systolic pressure to prevent discomfort for the patient. In an embodiment, the maximum pressure could be 90% of the systolic pressure. The pneumatic ring (114) inflates below the systolic pressure to enhance the flow of blood towards the ankle. Post an interval of 5 to 10 milliseconds after the pneumatic ring (114) is inflated, the system further inflates the bladder which in turn compresses the blood engrossed in the artery and pushes the blood into the arteries and capillaries.

FIG. 2 illustrates a system (200) and/or device having dual pistons to create a cyclical positive and negative pressure. The system (200) disclosed here is an extension of the system (100) of FIG. 1.

The system (200) disclosed here is an extension of the system (100) of FIG. 1. The system (200) of FIG. 2 employs two pistons, where the movement of one piston creates positive pressure of the cycle while the movement of another piston creates negative pressure of the cycle.

The system (200) is operatively coupled to a pneumatic chamber assembly (201). The pneumatic chamber assembly (201) further includes three components: a chamber (202), a limb inlet (203-A) and a pneumatic assembly inlet (203-B). The limb inlet (203-A) is adapted to receive a part of the limb. The pneumatic chamber assembly (201) is configured to sense the positive and negative pressure enacted by a first piston assembly (204) and a second piston assembly (208).

A first reservoir (104-A) is coupled to a first piston assembly (204), which further includes a first piston (206) where positive pressure is achieved by the forward movement of the first piston (206). The positive pressure is sensed by the pneumatic chamber assembly (201).

A second reservoir (104-B) is coupled to a second piston assembly (208), which further includes a second piston (210). A negative pressure is achieved by the backward movement of the second piston (210). The negative pressure is thus sensed by the pneumatic chamber assembly (201).

The system (200) having dual piston assemblies enact positive and negative pressure wherein, the first piston assembly (204) moves in forward direction, towards the limb, to enact positive pressure whereas the second piston assembly (208) moves in backward direction, away from the limb, to enact negative pressure. Thus, a cycle of alternate positive and negative pressure is achieved.

In an embodiment, distal part of a limb is placed between the pneumatic chamber assembly (201). A cycle of alternate positive and negative pressure is enacted by means of a first piston assembly (204) and second piston assembly (208). As the pressure on the chamber (202) increases, gap between the walls of pneumatic assembly inlet (203) is filled with air, thereby locking the walls, and increasing stiffness. Thus, the pressure on the limb is increased, keeping it steady. Thus, pneumatic chamber assembly (201) do not allow any leakage of air.

In another embodiment, the pneumatic assembly inlet (203-B) which enables sealing of the chamber (202) is a thin membrane with overhangs on the inside of the chamber (202) and outside of the chamber (202). As the chamber (202) is filled with air, creating positive pressure within the chamber (202), the section of the film overhanging inside the chamber (202) is compressed against the limb thus enhancing the seal and preventing any air leaks. When negative pressure is generated in the chamber (202), the section of the film outside the chamber (202) is compressed against the limb by the atmospheric pressure thus enhancing the seal from the outside and preventing any loss of negative pressure in the chamber (202). Thus, pneumatic chamber assembly (201) do not allow any leakage of air.

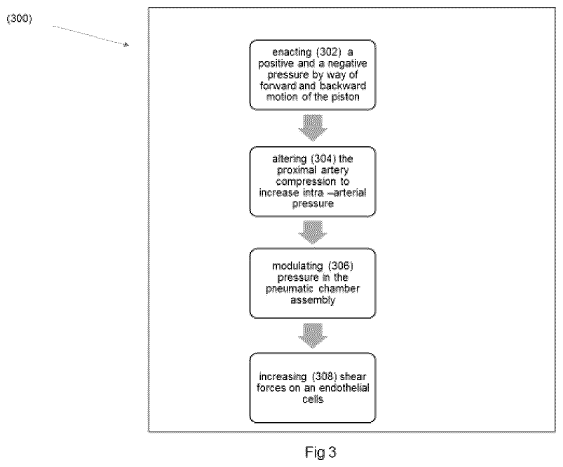

FIG. 3 illustrates a flowchart that depicts the method (300) for treating a damaged tissue by providing positive and negative pressure. The method (300) includes the piston-based systems/devices as described in FIG. 1 and FIG. 2.

The method (300) includes treating a damaged tissue by providing positive and negative pressure. The method (300) includes enacting (302) positive pressure by the action of piston (108) inside the tube (107). The method (300) further includes, altering (304) proximal artery compression, followed by modulating (306) pressure in a pneumatic chamber assembly (201) to increase intra-arterial pressure. The method (300) further includes, increasing (308) shear forces on an endothelial cell to remodel the endothelium and vasculature. The negative pressure increases intra-arterial pressure which gets transmitted forward through to the capillary system. The positive pressure increases the interstitial pressure and pushes out the interstitial fluid via the venous and lymphatic system, thus decreasing edema.

In an embodiment, the method (300) includes treating a damaged tissue by providing positive pressure. The method (300) includes enacting (302) positive pressure by the action of piston (108) inside the tube (107). The method (300) further includes, constricting (304) a proximal part of an artery, followed by compressing (306) a pneumatic chamber assembly (201) to increase intra-arterial pressure. The method (300) further includes, increasing (308) shear forces on an endothelial cell to remodel the endothelium and vasculature.

In another embodiment, the method (300) includes treating a damaged tissue by providing negative pressure. The method (300) includes enacting (302) negative pressure by the action of piston (108) inside the tube (107). The method (300) further removes, constricting (304) a proximal part of an artery, followed by negative pressure (306) in the pneumatic chamber assembly (201) to increase intra-arterial pressure. The method (300) further includes, increasing (308) shear forces on an endothelial cell to remodel the endothelium and vasculature.

Further, after the peak arterial pulse flow passes beneath the pneumatic ring, positive pressure compresses the pneumatic ring to compress the artery, following which chamber compression lead to maximum blood flow in the parts distal to the pneumatic ring.

In an embodiment, the method (300) is provided for regaining elasticity of the artery. In an embodiment, the method (300) is provided for treatment of a Raynaud's phenomenon/disease. In this condition the artery respond by constructing severely (spasm) when exposed to cold, this leads to decrease in the blood flow causing vascular damage to the limb. The negative pressure in the chamber increases intra-arterial pressure which gets transmitted forward through to the capillary system thus breaking the spasm. The positive pressure increases the interstitial pressure and pushes out the interstitial fluid via the venous and the lymphatic system, thus decreasing post ischemia edema. Decreased edema and increased oxygenated blood flow can improve tissue healing and decreases pain.

In another embodiment, the method (300) is provided for increasing oxygen transfer rate in the artery. An affected artery will provide an inverted V-shaped pressure curve. The methods and systems herein convert the inverted V-shaped pressured curves to inverted U-shaped curves. By having an inverted U-shape instead of inverted V-shape, the area under the curve where exchange of oxygen is going to take place will lead to higher oxygen transfer rate in comparison.

In another embodiment, the method (300) is provided for relieving arterial stiffness. Arterial stiffness is the major cause of morbidity and mortality in cardiovascular disease. In an embodiment, the method (300) for vascular remodeling of a tissue/artery is provided. The method (300) includes providing cyclical positive and negative pressures on an affected vessel. The cyclical pattern will decrease stiffness and enhance elasticity of the artery.

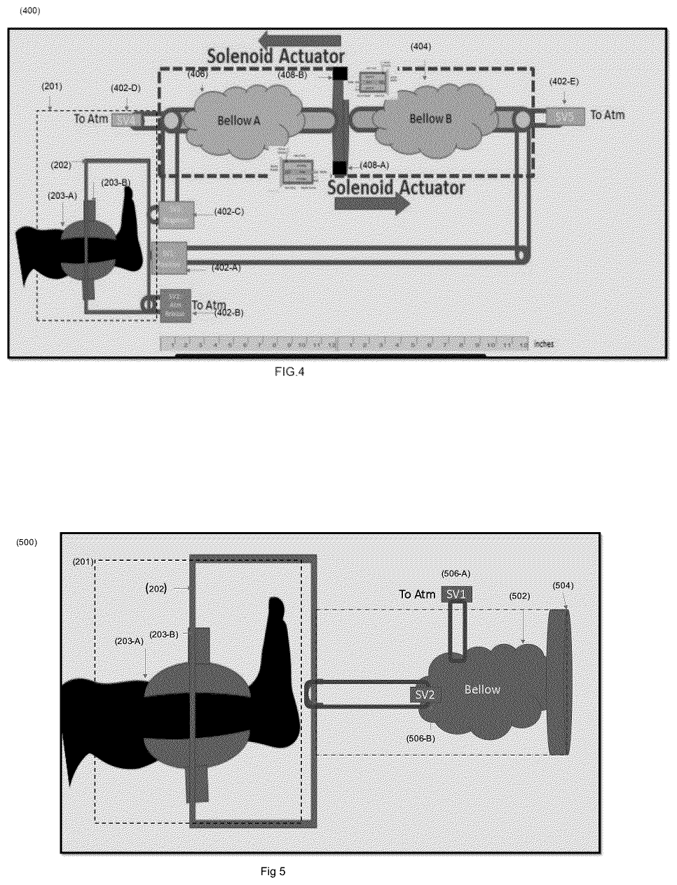

FIG. 4 illustrates a system (400) and/or device having an assembly of two bellows to create a cyclical positive and negative pressure.

The system (400) comprises an assembly of bellows coupled to a plurality of solenoid actuators to enact cyclic positive and negative pressure.

A first bellow (404) is coupled to a first solenoid actuator (408-A) whereas a second bellow (406) is coupled to a second solenoid actuator (408-B).

The system (400) enacts alternate positive and negative pressure, when the first solenoid actuator (408-A) pressurizes the first bellow (504), the first bellow (404) is compressed, thereby enacting positive pressure, whereas when the second solenoid actuator (408-B) decompresses the second bellow (406), the second below (406) elongates, thereby enacting negative pressure. Thus, a cycle of alternate positive and negative pressure is achieved.

Thus, the positive and negative pressure enacted by the first bellow (406) and second bellow (408) is sensed by the pneumatic chamber assembly (201).

FIG. 5 illustrates a system (500) and/or device having a single bellow to create a cyclical positive and negative pressure.

The system (500) includes a bellow (502), a solenoid actuator (504), a first solenoid valve (506-A) and a second solenoid valve (506-B).

The bellow (502) is controlled by the solenoid actuator (504). The excess pressure is released into the atmosphere via the first solenoid valve (506-A) whereas the positive and negative pressure enacted by the system (500) is sensed by the pneumatic chamber assembly (201).

In an embodiment, the solenoid actuator (504), compresses the bellow (502), bellow (502) contracts, thereby enacting positive pressure, whereas when the solenoid actuator (504) decompresses the bellow (502), the below (502) elongates, thereby enacting negative pressure. Thus, a cycle of positive and negative pressure is achieved.

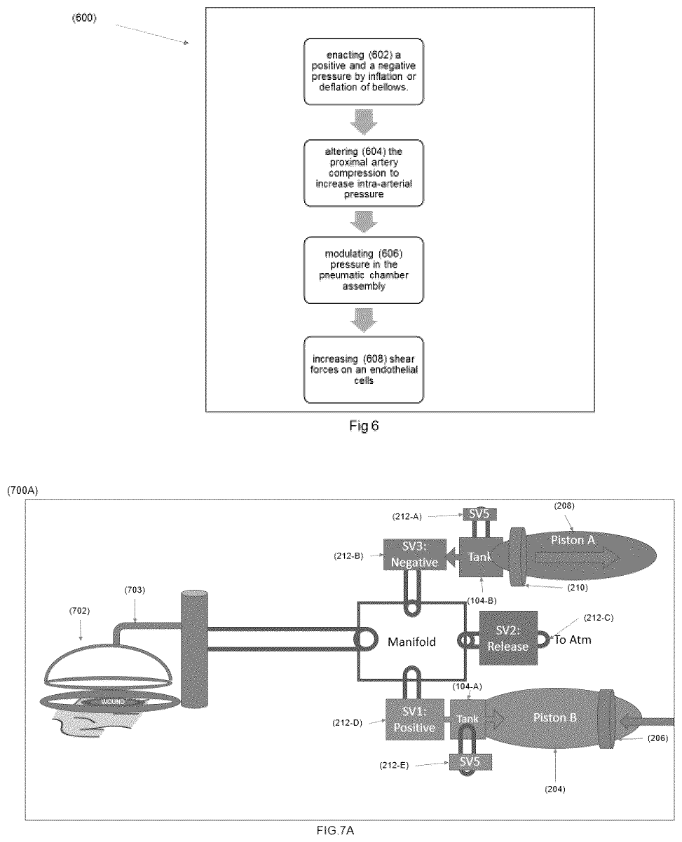

FIG. 6 illustrates a flowchart that depicts the method (600) for treating a damaged tissue by providing positive and negative pressure. The method (600) includes the bellow systems/devices as described in FIG. 5.

The method (600) includes treating a damaged tissue by providing positive and negative pressure. The method (600) includes enacting (602) a positive and negative pressure by a bellow (502). The method (600) further includes altering (604) a proximal artery compression to increase intra-arterial pressure, followed by modulating (606) pressure in the pneumatic chamber assembly (201). The method (600) further includes, increasing (608) shear forces on the endothelial cells to remodel the endothelium and vasculature.

In an embodiment, the method (600) includes treating a damaged tissue by providing positive pressure. The method (600) includes enacting (602) enacting positive pressure by the below (502). The method (600) further includes constricting (604) a proximal part of an artery, followed by compressing (606) a pneumatic chamber assembly (201) to increase intra-arterial pressure. The method (600) further includes, increasing (308) shear forces on an endothelial cell to remodel the endothelium and vasculature.

In another embodiment, the method (600) includes treating a damaged tissue by providing negative pressure. The method (600) includes enacting (602) negative pressure by the below (502). The method (600) further removes, constricting (604) a proximal part of an artery, followed by negative pressure (606) in the pneumatic chamber assembly (201) to increase intra-arterial pressure. The method (600) further includes, increasing (608) shear forces on an endothelial cell to remodel the endothelium and vasculature.

In an embodiment, the method (600) is provided for regaining elasticity of the artery. In an embodiment, the method (600) is provided for treatment of a Raynaud's phenomenon/disease. In an embodiment, the method (600) is provided for treatment of arterial stiffness.

In another embodiment, the method (600) is provided for increasing oxygen transfer rate in the artery. An affected artery will provide an inverted V-shaped pressure curve. The methods and systems herein convert the inverted V-shaped pressured curves to inverted U-shaped curves. By having an inverted u-shape instead of inverted V-shape, the area under the curve where exchange of oxygen is going to take place will lead to higher oxygen transfer rate in comparison.

In another embodiment, the method (600) is provided for relieving arterial stiffness. In an embodiment, the method (600) for vascular remodeling of a tissue/artery is provided. The method (600) includes providing cyclical positive and negative pressures on an affected vessel.

In another embodiment, the method (300) is provided for relieving arterial stiffness. Arterial stiffness is the major cause of morbidity and mortality in cardiovascular disease. In an embodiment, the method (300) for vascular remodeling of a tissue/artery is provided. The method (300) includes providing cyclical positive and negative pressures on an affected vessel. The cyclical pattern will decrease stiffness and enhance elasticity of the artery.

In an embodiment, the rest time in terms of heart beats between each cycle, the ratio should start from 1:5, 1:4 and then 1:2 where the second number in the ratio denotes the recovery time and the first number denotes the treatment followed by the number of beats for recovery. The ratio can repeat for a more than once before escalating to next ratio, for example 1:5, 1:5, 1:5; then 1:4, 1:4, 1:4 and then 1:2, 1:2 and 1:2. In an embodiment, the ratio can be a combination of various ratios in a programmed sequence.

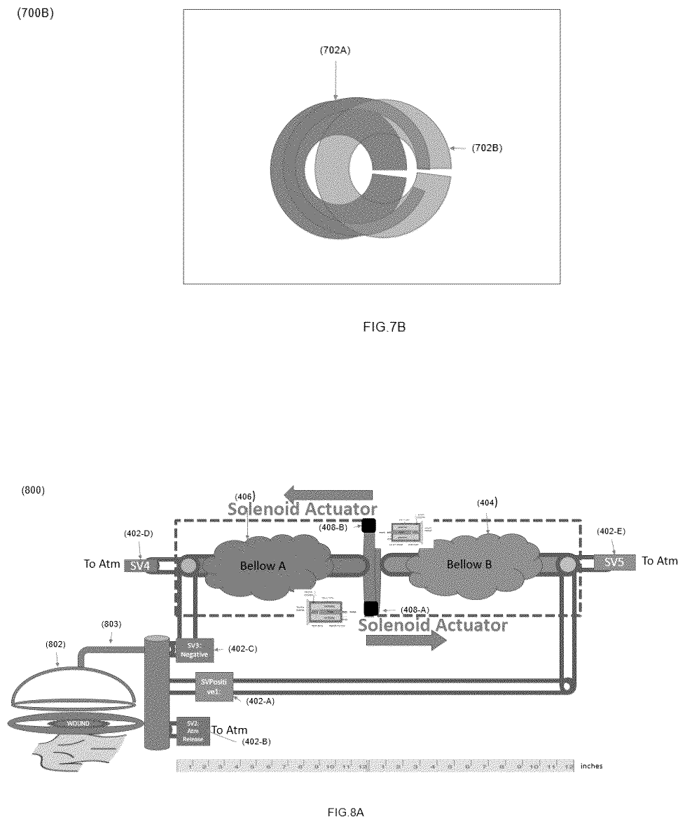

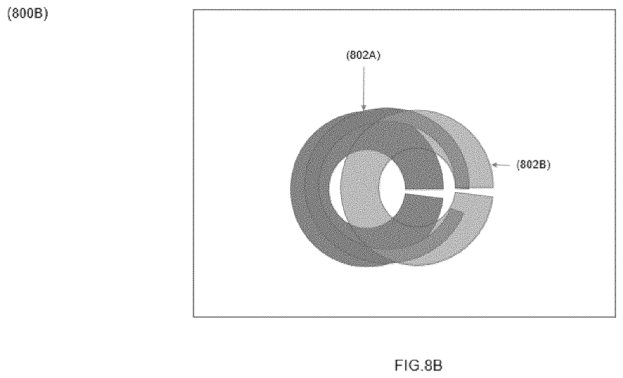

FIG. 7 illustrates a system (700) and/or device having dual pistons to create a cyclical positive and negative pressure. The system (700) disclosed herein is an extension of the system (200) of FIG. 2.

The system (700) of FIG. 7 employs two pistons, where the movement of one piston creates positive pressure of the cycle while the movement of another piston creates negative pressure of the cycle.

The system (700) is operatively coupled to a dome shaped pneumatic chamber assembly (701). The pneumatic chamber assembly (701) further includes two components: a chamber (702), and a pneumatic assembly inlet (703). The pneumatic chamber assembly (701) is configured to sense the positive and negative pressure enacted by a first piston assembly (204) and a second piston assembly (208).

In an embodiment, the chamber (702) further includes a holding plate (702-A) and a pneumatic ring (702-B) as illustrated in FIG. 7B,

A first reservoir (204-A) is coupled to a first piston assembly (204), which further includes a first piston (206) where positive pressure is achieved by the forward movement of the first piston (206). The positive pressure is sensed by the pneumatic chamber assembly (701).

A second reservoir (204-B) is coupled to a second piston assembly (208), which further includes a second piston (210). A negative pressure is achieved by the backward movement of the second piston (210). The negative pressure is thus sensed by the pneumatic chamber assembly (701).

The system (700) having dual piston assemblies enact positive and negative pressure wherein, the first piston assembly (704) moves in forward direction, towards the tissue, to enact positive pressure whereas the second piston assembly (708) moves in backward direction, away from the tissue, to enact negative pressure. Thus, a cycle of alternate positive and negative pressure is achieved.

In an embodiment, the pneumatic chamber assembly (701) is a dome shaped assembly provided for treatment of bed sores. The pneumatic assembly is placed over an ulcer in such a way that, a holding plate (702-A) pushes the skin inside the dome while the pneumatic ring (702-B) holds the skin and modulates the positive and negative pressure moving under the holding plate (702-A). The pneumatic ring (702-B) decreases the pressure over the skin to avoid damaging the ulcer or the surrounding skin. The dome shaped of the assembly forms a chamber for rotating the positive and negative pressure provided to the system.

In another embodiment, a method for treating an edema is provided. The method includes placing a pneumatic chamber assembly (701) on the area with edema. The method further includes enacting a positive and a negative pressure by way of forward and backward motion of a first piston (208) and a second piston assembly (210), wherein the forward movement of the first piston assembly (208) enacts a positive pressure, and the backward movement of the second piston assembly (210) enacts a negative pressure in a pneumatic chamber assembly (701). The method further includes modulating pressure in pneumatic chamber assembly (701) based on pulse cycle of a subject followed by increasing shear forces on an endothelial cell to remodel the endothelium and vasculature to treat the damaged tissue.

In an embodiment, the method includes the dome shaped pneumatic assembly of FIG. 7 and FIG. 8 The pneumatic assembly is placed over the edema in such a way that, a holding plate (702-A) pushes the skin inside the dome while the pneumatic ring (702-B) holds the skin and modulates the positive and negative pressure moving under the holding plate (702-A). The pneumatic ring (702-B) decreases the pressure over the skin to avoid damaging the edema or the surrounding skin, thus forming a chamber for rotating the positive and negative pressure provided to the system.

In an embodiment, the holding plate (702-A) may be glued to the skin without the pneumatic ring (702-B) to prevent pressure loss.

In an embodiment, the method for treating edema is timed with pulse wave cycle of a subject. With negative pressure in the dome at the time of arterial phase of the pulse, the blood will flow more towards the arterial system including capillaries. The positive pressure in the dome at the time of venous phase of the pulse, increases the interstitial tissue pressure and pushes the fluid out from the interstitial tissue into the venous and lymphatic system, thus removing the fluid from the edematous tissue and thus decreasing edema. Decreased edema and increased oxygenated blood flow circulation to the tissue can improve tissue healing and decreases pain.

In an embodiment, the method is provided for treating edema caused by renal and heart failure. In another embodiment, the method is provided for treating edema caused after cancer surgeries requiring lymphatic dissection like breast cancer.

In an embodiment, the method is provided for treating edema, selected from a group, cerebral edema, angioedema, hereditary angioedema, papilledema, macular edema, dependent edema, scrotal lymphedema, lipedema.

In another embodiment, the method for treating a bed sore is provided. The method includes placing a pneumatic chamber assembly (701) on the bed sore and enacting a positive and a negative pressure by way of forward and backward motion of a first piston assembly (208) and the second piston assembly (210) wherein the forward movement of the first piston assembly (208) enacts a positive pressure, and the backward movement of the second piston assembly (210) enacts a negative pressure in a pneumatic chamber assembly (701). The method further includes modulating pressure in pneumatic chamber assembly (701) based on pulse cycle of a subject followed by increasing shear forces on an endothelial cell to remodel the endothelium and vasculature to treat the damaged tissue.

In an embodiment, the method includes the dome shaped pneumatic assembly of FIG. 7. The pneumatic assembly is placed over the bed sore in such a way that, a holding plate pushes the skin inside the dome while the pneumatic ring holds the skin and modulates the positive and negative pressure moving under the holding plate. The pneumatic ring decreases the pressure over the skin to avoid damaging the bed sore or the surrounding skin, thus forming a chamber for rotating the positive and negative pressure provided to the system.

In an embodiment, the pneumatic dome assembly can be placed upside down inside a bed to enact pressure modulation to the diseased area while the patient is lying on his back.

In another embodiment, the method is provided to treat ulcers. In an embodiment, the method is provided to treat ulcers present on mucosal surfaces.

FIG. 8 illustrates a system (800) and/or device having an assembly of two bellows to create a cyclical positive and negative pressure. The system (800) is a further extension of FIG. 4.

The system (800) comprises an assembly of bellows coupled to a plurality of solenoid actuators to enact cyclic positive and negative pressure.

A first bellow (404) is coupled to a first solenoid actuator (408-A) whereas a second bellow (406) is coupled to a second solenoid actuator (408-B).

The system (800) enacts alternate positive and negative pressure, when the first solenoid actuator (408-A) compresses the first bellow (404), thereby enacting positive pressure, whereas when the second solenoid actuator (408-B) decompresses the second bellow (406), the second below (406) elongates, thereby enacting negative pressure. Thus, a cycle of alternate positive and negative pressure is achieved.

Thus, the positive and negative pressure enacted by the first bellow (406) and second bellow (408) is sensed by the pneumatic chamber assembly (801). The pneumatic chamber assembly further includes: a chamber (802) and pneumatic assembly inlet (803). In an embodiment, the pneumatic chamber assembly (801) is dome shaped.

In an embodiment, the chamber (802) further includes a holding plate (702-A) and a pneumatic ring (802-B) as illustrated in FIG. 8B,

In an embodiment, the dome shaped assembly is made of stiff transparent material, including glass, plastic, acrylic, polycarbonate and cellophane. In another embodiment, the dome shape is obtained by inflating a two layered flexible material dome, which once inflated, takes the shape of a stiff dome and thus provides stiffness at the time of pressure cycling, thus preventing pressure loss due to compliant dome.

In another embodiment, a method for treating an edema is provided. The method includes placing a pneumatic chamber assembly (801) on the area with edema. The method further includes enacting a positive and negative pressure, wherein the first solenoid actuator (408-A) compresses the first bellow (404) to enact positive pressure, and the second solenoid actuator (408-B) decompresses the second bellow (406) to enact negative pressure. The method further includes modulating pressure in pneumatic chamber assembly (801) based on pulse cycle of a subject followed by increasing shear forces on an endothelial cell to remodel the endothelium and vasculature to treat the damaged tissue.

In an embodiment, the method includes the dome shaped pneumatic assembly of FIG. 8. The pneumatic assembly is placed over the edema in such a way that, a holding plate (802-A) pushes the skin inside the dome while the pneumatic ring (802-B) holds the skin and modulates the positive and negative pressure moving under the holding plate (802-A). The pneumatic ring (802-B) decreases the pressure over the skin to avoid damaging the edema or the surrounding skin, thus forming a chamber for rotating the positive and negative pressure provided to the system.

In an embodiment, the holding plate (802-A) may be glued to the skin without the pneumatic ring (802-B) to prevent pressure loss.

In an embodiment, the method for treating edema is timed with pulse wave cycle of a subject. With negative pressure in the dome at the time of arterial phase of the pulse, the blood will flow more towards the arterial system including capillaries. The positive pressure in the dome at the time of venous phase of the pulse, increases the interstitial tissue pressure and pushes the fluid out from the interstitial tissue into the venous and lymphatic system, thus removing the fluid from the edematous tissue and thus decreasing edema. Decreased edema and increased oxygenated blood flow circulation to the tissue can improve tissue healing and decreases pain.

In an embodiment, the method is provided for treating edema caused by renal and heart failure. In another embodiment, the method is provided for treating edema caused after cancer surgeries requiring lymphatic dissection like breast cancer.

In an embodiment, the method is provided for treating edema, selected from a group, cerebral edema, angioedema, hereditary angioedema, papilledema, macular edema, dependent edema, scrotal lymphedema, lipedema.

In another embodiment, the method for treating a bed sore is provided. The method includes placing a pneumatic chamber assembly (801) on the bed sore and enacting a positive and negative pressure, wherein the first solenoid actuator (408-A) compresses the first bellow (404) to enact positive pressure, and the second solenoid actuator (408-B) decompresses the second bellow (406) to enact negative pressure. The method further includes modulating pressure in pneumatic chamber assembly (801) based on the pulse cycle of a subject followed by increasing shear forces on an endothelial cell to remodel the endothelium and vasculature to treat the damaged tissue.

In an embodiment, the method includes the dome shaped pneumatic assembly of FIG. 8. The pneumatic assembly is placed over the bed sore in such a way that, a holding plate pushes the skin inside the dome while the pneumatic ring holds the skin and modulates the positive and negative pressure moving under the holding plate. The pneumatic ring decreases the pressure over the skin to avoid damaging the bed sore or the surrounding skin, thus forming a chamber for rotating the positive and negative pressure provided to the system.

In an embodiment, the pneumatic dome assembly can be placed upside down inside a bed to enact pressure modulation to the diseased area while the patient is lying on his back.

In another embodiment, the method is provided to treat ulcers. In an embodiment, the method is provided to treat ulcers present on mucosal surfaces.

Certain terms are used throughout the following description and claims to refer to particular features or components. As one skilled in the art will appreciate, different persons may refer to the same feature or component by different names. This document does not intend to distinguish between components or features that differ in name but not structure or function. While various embodiments of the present disclosure have been illustrated and described, it will be clear that the present disclosure is not limited to these embodiments only. Numerous modifications, changes, variations, substitutions, and equivalents will be apparent to those skilled in the art, without departing from the spirit and scope of the present disclosure, as described in the claims.

1. A system (100) for enacting a positive and a negative pressure on a limb, the system (100) comprises:
a) a reservoir (104) to entrap air;
b) an air compressor (102) coupled to the reservoir (104) to entrain atmospheric air;
c) a piston assembly (106) coupled with the reservoir (104) to enact positive and negative pressure, wherein a forward movement of the piston (108) enacts a positive pressure, and a backward movement of the piston (108) enacts a negative pressure; and
d) a pneumatic ring (114), placed over a portion of the limb, coupled to the system (100) via the piston assembly (106) that maintains intermittent pressure to prevent formation of a venous blood clot and ischemic injury to a tissue distal to the pneumatic ring (114) compression due to lack of blood flow.

2. The system (100) of claim 1, wherein the pressure measuring mechanism is selected from a group, but not limited to, a pressure gauge, barometer, piezometer, pressure tube, manometer, bourdon gauge, diaphragm pressure gauge, micro manometer.

3. The system (100) of claim 1, wherein the pressure enacted by the system is sensed by the pneumatic ring (114) placed over a portion of the limb.

4. The system (100) of claim 1, further comprising a dual piston assembly to enact positive and negative pressure on the limb with one or both of a first piston assembly (204) and a second piston assembly (208), wherein the system (200) comprises:
a) the air compressor (102) coupled to the reservoir (104) to entrain atmospheric air;
b) a pneumatic chamber assembly (201) coupled to the system (100) maintains intermittent pressure to prevent formation of a venous blood clot;
c) the first piston assembly (204) further comprising a first piston (206);
d) the second piston assembly (208) comprising a second piston (210);
enacting positive and negative pressure, wherein forward movement of the first piston (206), towards the limb, enacts positive pressure and the backward movement of the second piston (210), away from the limb, enacts negative pressure.

5. The system (200) of claim 4, the system (200) further comprises a plurality of reservoirs, wherein the first piston assembly (204) is connected to a first reservoir (104-A) and the second piston assembly (208) is connected to a second reservoir (104-B).

6. The system (200) of claim 4, wherein the first piston assembly (204) moves in forward direction, towards the limb, to enact positive pressure whereas the second piston assembly (208) moves in backward direction, away from the limb, to enact negative pressure.

7. The system (200) of claim 4, wherein the positive and negative pressure enacted by the system (200) is sensed by the pneumatic chamber assembly (201) placed over a portion of the limb via plurality of solenoid valves.

8. The system (200) of claim 4, wherein the pressure enacted by the system (200) is sensed by the pneumatic chamber assembly (201), through a first solenoid valve (212-D) and the excess pressure is released into the environment through a second solenoid valve (212-B).

9. A method (300) for treating a damaged tissue, the method (300) comprises:
a) enacting (302) a positive and a negative pressure by way of forward and backward motion of a piston (108),
wherein the forward movement of the piston (108) enacts a positive pressure, and the backward movement of the piston (108) enacts a negative pressure in a pneumatic chamber assembly (201);
b) altering (304) a proximal artery compression to increase intra-arterial pressure; and
c) modulating (306) pressure in pneumatic chamber assembly (201); and
d) increasing (308) shear forces on an endothelial cell to remodel the endothelium and vasculature to treat the damaged tissue.

10. The method (300) of claim 9, wherein the method (300) further comprises a step for relieving the arterial stress by increasing dilation in the artery.

11. The method (300) of claim 9, wherein the pressure is increased to increase dilation in the artery, thereby increasing the oxygen transfer rate in an arterial system.

12. A system (500) for enacting a positive and a negative pressure on a limb, the system (500) comprises:
a) a bellow (502) coupled to a solenoid actuator (504) to enact positive and negative pressure;
b) a pneumatic chamber assembly (201) coupled to the system (500) to maintain intermittent pressure to prevent formation of a venous blood clot; and
wherein the solenoid actuator (504) compresses the bellow (502) to enact positive pressure, and the solenoid actuator (504) decompresses the bellow (504) to enact negative pressure.

13. The system (500) of claim 12, wherein the pressure enacted by the system (500) is sensed by the pneumatic chamber assembly (201) via a first solenoid valve (506-B) and the excess pressure is released into the environment via a second solenoid valve (506-A).

14. The system (500) of claim 12, further comprises an assembly of bellows to enact positive and negative pressure on the limb, with one or both of a first bellow (404) and a second bellow (406), wherein the system (500) comprises:
a) the first bellow (404) is connected to a first solenoid actuator (408-A) and the second bellow (406) is connected to a second solenoid actuator (408-B);
b) a pneumatic chamber assembly (201) coupled to the system (500) maintains intermittent pressure to prevent formation of a venous blood clot;
enacting a positive and negative pressure, wherein the first solenoid actuator (408-A) compresses the first bellow (404) to enact positive pressure, and the second solenoid actuator (408-B) decompresses the second bellow (406) to enact negative pressure.

15. A method (600) for treating a damaged tissue, the method (600) comprises:
a) enacting (602) a positive and negative pressure by the action of a bellow, wherein a solenoid actuator (504) compresses the bellow (502) to enact positive pressure, and the solenoid actuator (504) decompresses the bellow (504) to enact negative pressure.
b) altering (604) a proximal artery compression to increase intra-arterial pressure; and
c) modulating (606) pressure in a pneumatic chamber assembly (201) to increase intra-arterial pressure;
d) increasing (608) shear forces on an endothelial cell to remodel the endothelium and vasculature to treat the damaged tissue.

16. A system (200) for enacting a positive and a negative pressure on a limb, with one or both of a first piston assembly (204) and a second piston assembly (208), wherein the system (200) comprises:
a) an air compressor (102) coupled to a reservoir (104) to entrain atmospheric air;
b) a pneumatic chamber assembly (201) coupled to the system (100) maintains intermittent pressure and prevents formation of a venous blood clot;
c) the first piston assembly (204) comprising a first piston (206);
d) the second piston assembly (208) comprising a second piston (210);
enacting a positive and a negative pressure, wherein a forward movement of the first piston (206), towards the limb, enacts positive pressure and the backward movement of the second piston (208), away from the limb, enacts negative pressure.

17. A system (400) for enacting a positive and a negative pressure on a limb, with one or both of a first bellow (404) and a second bellow (406), wherein the system (400) comprises:
a) the first bellow (404) is coupled to a first solenoid actuator (408-A) and the second bellow (406) is coupled to a second solenoid actuator (408-B);
b) a pneumatic chamber assembly (201) coupled to the system (500) maintains intermittent pressure and prevents formation of a blood clot;
enacting a positive and negative pressure, wherein the first solenoid actuator (408-A) compresses the first bellow (404) to enact positive pressure, and the second solenoid actuator (408-B) decompresses the second bellow (406) to enact negative pressure.

18. A system (700) for enacting a positive and a negative pressure on a skin, with one or both of a first piston assembly (204) and a second piston assembly (208), wherein the system (200) comprises:
a) an air compressor (102) coupled to a reservoir (104) to entrain atmospheric air;
b) a pneumatic chamber assembly (701) coupled to the system (700) maintains intermittent pressure and prevents formation of a venous blood clot;
c) the first piston assembly (204) comprising a first piston (206);
d) the second piston assembly (208) comprising a second piston (210);
enacting a positive and a negative pressure, wherein a forward movement of the first piston (206), towards the skin, enacts positive pressure and the backward movement of the second piston (210), away from the skin, enacts negative pressure.

19. The system (700) of claim 18, wherein the system (700) further comprises a plurality of reservoirs, wherein the first piston assembly (204) is connected to a first reservoir (104-A) and the second piston assembly (208) is connected to a second reservoir (104-B).

20. The system (700) of claim 18, wherein the first piston assembly (204) moves in forward direction, towards the skin, to enact positive pressure whereas the second piston assembly (208) moves in backward direction, away from the skin, to enact negative pressure.

21. The system (700) of claim 18, wherein the positive and negative pressure enacted by the system (700) is sensed by the pneumatic chamber assembly (701) placed over a portion of the skin via plurality of solenoid valves.

22. The system (700) of claim 18, wherein the positive pressure is sensed by the pneumatic chamber assembly (701), through a first solenoid valve (212-D) and the negative pressure is sensed by the pneumatic chamber assembly (701), through a second solenoid valve (212-B).

23. A method for treating edema using the system of claim 18, the method comprises:
a) placing a pneumatic chamber assembly (701) on the edema;
b) enacting a positive and a negative pressure by way of forward and backward motion of a first piston (206) and a second piston (210),
wherein the forward movement of the first piston (206) enacts a positive pressure, and the backward movement of the second piston (210) enacts a negative pressure in a pneumatic chamber assembly (701);
c) modulating pressure in pneumatic chamber assembly (701) based on pulse cycle of a subject; and
d) increasing shear forces on an endothelial cell to remodel the endothelium and vasculature to treat the damaged tissue.

24. A method for treating a bed sore using the system of claim 18, the method comprises:
a) placing a pneumatic chamber assembly (701) on the bed sore;
b) enacting a positive and a negative pressure by way of forward and backward motion of a first piston (206) and a second piston (210),
wherein the forward movement of the first piston (206) enacts a positive pressure, and the backward movement of the second piston (210) enacts a negative pressure in a pneumatic chamber assembly (701);
c) modulating pressure in pneumatic chamber assembly (701) based on pulse cycle of a subject; and
d) increasing shear forces on an endothelial cell to remodel the endothelium and vasculature to treat the damaged tissue.

25. A system (800) for enacting a positive and a negative pressure on a skin, with one or both of a first bellow (404) and a second bellow (406), wherein the system (800) comprises:
a) the first bellow (404) is coupled to a first solenoid actuator (408-A) and the second bellow (406) is coupled to a second solenoid actuator (408-B);
b) a pneumatic chamber assembly (801) coupled to the system (800) maintains intermittent pressure and prevents formation of a venous blood clot;
enacting a positive and negative pressure, wherein the first solenoid actuator (408-A) compresses the first bellow (404) to enact positive pressure, and the second solenoid actuator (408-B) decompresses the second bellow (406) to enact negative pressure.

26. The system (800) of claim 25, wherein the pressure enacted by the system (800) is sensed by the pneumatic chamber assembly (801) via a first solenoid valve (506-B) and the excess pressure is released into the environment via a second solenoid valve (506-A).

27. A method for treating edema using the system of claim 25, the method comprises:
a) placing a pneumatic chamber assembly (801) on the edema;
b) enacting a positive and negative pressure, wherein the first solenoid actuator (408-A) compresses the first bellow (404) to enact positive pressure, and the second solenoid actuator (408-B) decompresses the second bellow (406) to enact negative pressure;
c) modulating pressure in pneumatic chamber assembly (201) based on pulse cycle of a subject; and
d) increasing shear forces on an endothelial cell to remodel the endothelium and vasculature to treat the damaged tissue.

28. A method for treating a bed sore using the system of claim 25, the method comprises:
a) placing a pneumatic chamber assembly (801) on the bed sore;
b) enacting a positive and negative pressure, wherein the first solenoid actuator (408-A) compresses the first bellow (404) to enact positive pressure, and the second solenoid actuator (408-B) decompresses the second bellow (406) to enact negative pressure;
c) modulating pressure in pneumatic chamber assembly (801) based on pulse cycle of a subject; and
d) increasing shear forces on an endothelial cell to remodel the endothelium and vasculature to treat the damaged tissue.
